(19) United States
(12) Patent Application Publication
(10) Pub. No.: US 2021/0120749 A1
(43) Pub. Date: Apr. 29, 2021

(54) ARTIFICIAL INOCULATION METHOD FOR BACTERIAL PANICLE BLIGHT OF RICE

(71) Applicant: China National Rice Research Insititute, Hangzhou (CN)

(72) Inventors: Yuxuan HOU, Hangzhou (CN); Shiwen HUANG, Hangzhou (CN); Lianmeng LIU, Hangzhou (CN); Ling WANG, Hangzhou (CN)

(73) Assignee: China National Rice Research Insititute, Hangzhou (CN)

(21) Appl. No.: 17/015,044

(22) Filed: Sep. 8, 2020

(30) Foreign Application Priority Data
Oct. 23, 2019 (CN) 201911011760.1

(51) Int. Cl. A01G 7/06; A01G 22/22

(52) U.S. Cl. CPC A01G 7/06; A01G 22/22

(57) ABSTRACT

Provided is an artificial inoculation method for bacterial panicle blight of rice. The method includes the steps of S1. preparing an inoculum using a bacteria solution of Burkholderia glumae; S2. inoculating at the booting stage of rice; S3. adopting an inoculation method of injection, and injecting the inoculum prepared in the step S1 into the hollow booting part of a rice stalk from bottom to top by an injector; and S4. calculating the incidence rate and disease index after inoculation according to the number of diseased panicles and the number of diseased grains.

### TECHNICAL FIELD

The present invention belongs to a pathogenic bacteria inoculation method in the field of plant pathology, and particularly relates to an artificial inoculation method for bacterial panicle blight of rice.

### BACKGROUND

Bacterial panicle blight of rice (BPBR), also called bacterial grain rot of rice, is one of the world's rice diseases caused by Burkholderia glumae. The disease occurs in Asia, Africa and America, and causes wilting and rot of rice seedlings, and immature husk, browning and decay of grains, resulting in yield loss up to 80%.

In 2007, bacterial panicle blight of rice was listed as a quarantine disease in China. However, in recent years, due to the change in climate, the replacement of rice varieties (combinations), the change in farming and cultivation systems, and other factors, the disease is currently occurring and spreading in China, and has become a recently rapidly increasing bacterial disease. Therefore, it is a primary problem urgently to be solved in production about how to scientifically control panicle blight. The most effective method to control panicle blight is to cultivate disease-resistant varieties, and rice germplasm resources and variety resistance identification and evaluation are key factors. Among which, a fast, simple and efficient artificial inoculation method is a reliable and accurate guarantee for resistance identification.

There are two methods for resistance identification and evaluation of panicle blight: spontaneous induction and artificial inoculation. Due to the influence of bacterial source cardinal in the field and temperature and humidity, spontaneous induction in the field may cause no incidence or insufficient incidence, and poor consistency and stability of identification, so the method is rarely used. The artificial inoculation method for panicle blight already reported at present is mainly the inoculation method of spraying of bacteria solution at the heading stage of rice (Mizobuchi1R. et al. Mapping a quantitative trait locus for resistance to bacterial grain rot in rice. Rice, 2013, 6:13): cutting panicles 3 days after heading, spraying 10⁸ cfu/mL bacteria solution, covering with a preservation bag, culturing with clean water, placing in an illumination incubator (first at 25° C. and relative humidity of 100%, 20 h; then at 27° C. and relative humidity greater than 80%; 14 h in light condition/10 h in dark condition), and observing the incidence condition after 6-7 days. However, it is found in the test that the method is not only time-consuming in moisturizing measures, but is low in inoculation efficiency, so the degree of incidence varies greatly, and the problem of no incidence or insufficient incidence after spraying occurs, seriously hindering the research on resistance to bacterial panicle blight of rice.

### SUMMARY

The present invention designs an efficient artificial inoculation method for bacterial panicle blight of rice.

To solve the above technical problem, the present invention adopts the following technical solution:

The artificial inoculation method for bacterial panicle blight of rice, comprising the following steps:

S1. preparing an inoculum using a bacteria solution of Burkholderia glumae;

S2. inoculating at the booting stage of rice, thereby being conductive to the colonization of pathogenic bacteria and incidence of disease;

S3. adopting an inoculation method of injection, and injecting the inoculum prepared in the step S1 into the hollow booting part of a rice stalk from bottom to top by an injector; and

S4. after inoculation, according to the number of diseased panicles and the number of diseased grains, recording the incidence condition of rice, and calculating the incidence rate and disease index.

The artificial inoculation method for bacterial panicle blight of rice, wherein the inoculum in the step S1 is acquired by the following steps:

S11. transferring standard strains LMG2196 of Burkholderia glumae frozen at −70° C. into a NA liquid medium containing 50 μg/mL ampicillin in a ratio of 1:1000;

S12. culturing for 16-24 h at 37° C. and 200 rpm/min; and

S13. diluting the bacteria solution cultured in the step S22 with sterile purified water until the OD600 value of the bacteria solution is 0.2-0.3.

The artificial inoculation method for bacterial panicle blight of rice, wherein in the step S3, the specific position of injection is 5-7 cm below the last leaf of rice.

The artificial inoculation method for bacterial panicle blight of rice, wherein in the step S3, the injection volume of the inoculum is 0-1 mL until the bacteria solution overflows from the top of the stalk.

The artificial inoculation method for bacterial panicle blight of rice, wherein in the step S3, the inoculation is performed in an environment where the average daily temperature is greater than 30° C. and the relative humidity is greater than 75%.

The artificial inoculation method for bacterial panicle blight of rice, wherein in the step S3, the inoculation is specifically performed between 16:00-18:00 in the evening.

The artificial inoculation method for bacterial panicle blight of rice, wherein the injection volume of the inoculum is 0.5 mL.

The artificial inoculation method for bacterial panicle blight of rice, wherein in the step S3, at least 3 tillers are injected for each rice plant.

The artificial inoculation method for bacterial panicle blight of rice has the following advantageous effects:

The present invention is simple, fast, efficient and safe, the statistical incidence rate reaches 100% after 3-4 weeks, and the disease index is up to 75 or more. The method is an efficient inoculation method for bacterial panicle blight of rice, can be widely used for the pathogenic typing of pathogenic bacteria of panicle blight, the identification of resistance of rice varieties, and the research on the interaction between pathogenic bacteria and rice, is conducive to screening out good varieties with high resistance, solves the problem of lack of varieties resistant to panicle blight in production at present, and has an important promotion effect on research of varieties resistant to bacterial panicle blight of rice.

### DESCRIPTION OF DRAWINGS

FIG. 1 shows the comparison between inoculation effects of two inoculation methods, wherein FIG. 1A: symptom of stalk injection at booting stage; FIG. 1B: symptom of inoculation of spraying at heading stage; FIG. 1C: incidence rates of two inoculation methods; and FIG. 1D: disease indexes of two inoculation methods; Variety: Yongyou; Culture manner: pot culture under natural conditions.

FIG. 2 shows the incidence condition of stalk injection at the booting stage, wherein FIG. 2A: symptom of stalk injection at booting stage in field; FIG. 2B: incidence rate; and FIG. 2C: disease index; Variety: Huazhan; Culture manner: field culture.

### DETAILED DESCRIPTION

In order to make the technical solution of the present invention easier to understand and master, the specific embodiments of the present invention will be described in further detail below with reference to the accompanying drawings.

An efficient artificial inoculation method for bacterial panicle blight of rice, comprising the following steps:

(1) Transferring strains LMG2196 of Burkholderia glumae frozen at −70° C. into a NA liquid medium containing 50 μg/mL ampicillin in a ratio of 1:1000, culturing for 16-24 h at 37° C. and 200 rpm/min, diluting the bacteria solution with sterile purified water until the OD600 value of the bacteria solution is 0.2-0.3, and using the diluted bacteria solution as an inoculum.

(2) About 16:00-17:00 in the afternoon, injecting about 0.5 mL of the prepared bacteria solution from bottom to top with a 1 mL of medical sterilized injector into the hollow booting part of the rice stalk (that is, the position about 5-7 cm below the last leaf) until the bacteria solution overflows from the top of the stalk.

(3) Growing the rice inoculated in the step (2) for 10-12 weeks (booting stage).

(4) Performing the inoculation operation in the step (2) in an environment where the average daily temperature is greater than 30° C. and the relative humidity is greater than 75%.

(5) Investigating the incidence condition 3-4 weeks after inoculation, and taking the number of diseased panicles and the number of diseased grains as investigation indicators.

(6) Calculating the incidence rate of bacterial panicle blight of rice, wherein the calculation formula of the incidence rate is: (number of diseased panicles/total number of inoculated panicles)*100%.

(7) Calculating the disease index of bacterial panicle blight of rice, wherein the calculation formula of the disease index is: [Σ(number of panicles of all levels*corresponding level number)/(total number of investigated panicles*highest level number)]*100.

See Table 1 for standards of disease levels.

TABLE 1
Standards of evaluation levels of bacterial panicle blight of rice

| Disease levels | Average diseased grain rate |
|---|---|
| Level 0 | Panicles are not discolored |
| Level 1 | 1-20% of grains are discolored |
| Level 3 | 21-40% of grains are discolored |
| Level 5 | 41-60% of grains are discolored |
| Level 7 | 61-80% of grains are discolored |
| Level 9 | More than 81% of grains are discolored |

### Embodiment 1

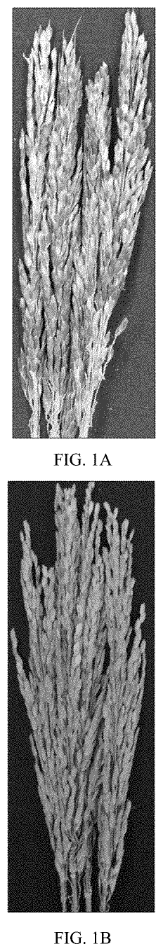

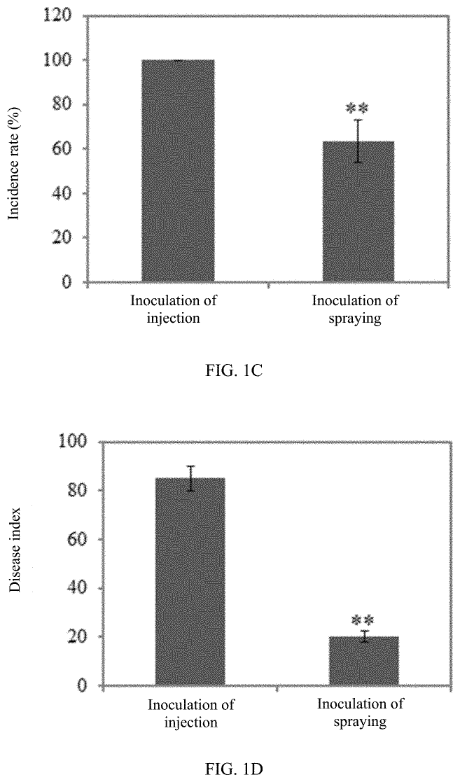

With reference to FIG. 1, rice of which the variety is Yongyou is planted, about May 20, Yongyou seedlings are transplanted into blue plastic pots after soaking and sowing 25-30 days as usual, wherein the plastic pots are 50 cm in length, 35 cm in width and 16 cm in height, 9 plants are transplanted in each pot, each variety is provided with three repeated samples, the plastic pots are placed outdoors under natural conditions for cultivation and management, and the plants are used for inoculation test when growing to the booting stage.

Preparation of bacteria solution for inoculation: transferring standard strains LMG2196 of Burkholderia glumae frozen at −70° C. into a NA liquid medium containing 50 μg/mL ampicillin in a ratio of 1:1000, culturing for 16-24 h at 37° C. and 200 rpm/min, diluting the bacteria solution with sterile purified water until the OD600 value of the bacteria solution is 0.2-0.3, and using the obtained bacteria solution A as an inoculum.

Inoculation of stalk injection at booting stage: about 10-12 weeks after sowing of Yongyou rice, that is, the booting stage, at 16:00-17:00 in the afternoon, selecting the hollow booting part of a rice stalk, that is, the position 5-7 cm below the last leaf, pressing against the back of the stalk by the index finger and middle finger of the left hand, and holding a 1 mL of medical injector by the right hand to slowly inject about 0.5 mL of bacteria solution A of LMG2196 from bottom to top in the direction parallel to the stalk until the bacteria solution overflows from the top of the stalk, wherein 3 tillers are injected for each plant, 27 tillers are injected for each pot, each variety is provided with three repeated samples, the pots are placed outdoors under natural conditions, and the incidence condition is observed every day and recorded in time.

Calculation of incidence rate and disease index: performing follow-up investigation on the incidence condition after inoculation of injection, about 4 weeks later, investigating the incidence rate of panicles and the discoloration ratio of grains (the percentage of red-brown grains per panicle to the total number of grains), and calculating the disease index according to the standards of levels in Table 1.

Incidence rate of panicles=(number of diseased panicles/total number inoculated panicles)*100%.

Disease index=[Σ(number of panicles of all levels*corresponding level number)/(total number of investigated panicles*highest level number)]*100.

Traditional inoculation of spraying at heading stage: 2-3 days after rice heading, spraying the bacteria solution for inoculation with an OD600 value of about 0.4 at 16:00-17:00 in the afternoon, the bacteria solution containing 0.01% of Tween 20, investigating the incidence condition after about 4 weeks, and calculating the incidence rate and disease index.

In this embodiment, about 4 weeks after inoculation of rice of which the variety is Yongyou with stalk injection at the booting stage for bacterial panicle blight of rice, the diseased panicles show typical symptoms of panicle blight: grain discoloration (red-brown disease spot), inadequate grain filling, immature husk (FIG. 1A), the incidence rate is 100% (FIG. 1C), the disease index is up to 85 (FIG. 1D), and LMG2196 of Burkholderia glumerea can be isolated again from diseased grains.

By adopting the traditional inoculation of spraying at the heading stage, the number of diseased grains (discolored and having immature husks) is less than that by adopting stalk injection at the booting stage of grains (FIG. 1B); the incidence rate is only 63%, and the disease index is only 20%, which are significantly lower than the incidence rate (100%) and the disease index (85) (FIG. 1C and FIG. 1D) of inoculation of stalk injection at the booting stage (P<0.01).

### Embodiment 2

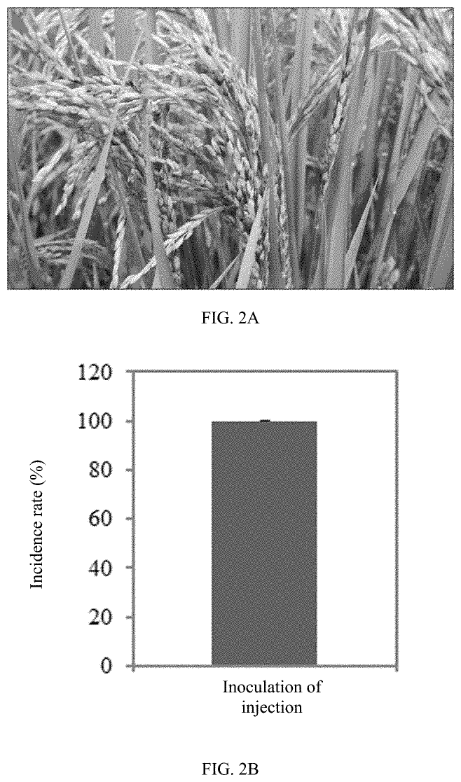

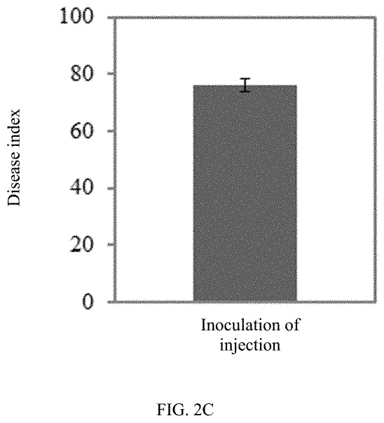

With reference to FIG. 2, rice of which the variety is Huazhan is planted, about May 20, Huazhan seedlings are transplanted into the field after soaking and sowing 25-30 days as usual, the seedlings are transplanted into 4 rows, with 6 seedlings in each row, wherein the row spacing is 20 cm, the plant spacing is 16 cm, each variety is provided with three repeated samples for conventional filed management, and the plants are used for inoculation test when growing to the booting stage.

Preparation of bacteria solution for inoculation: transferring standard strains LMG2196 of Burkholderia glumae frozen at −70° C. into a NA liquid medium containing 50 μg/mL ampicillin in a ratio of 1:1000, culturing for 16-24 h at 37° C. and 200 rpm/min, diluting the bacteria solution with sterile purified water until the OD600 value of the bacteria solution is 0.2-0.3, and using the obtained bacteria solution A as an inoculum.

Inoculation of stalk injection at booting stage: about 10 weeks after sowing of Huazhan, that is, the booting stage, at 16:00-17:00 in the afternoon, selecting the hollow booting part of a rice stalk, that is, the position 5-7 cm below the last leaf, pressing against the back of the stalk by the index finger and middle finger of the left hand, and holding a 1 mL of medical injector by the right hand to slowly inject about 0.5 mL of bacteria solution A of LMG2196 from bottom to top in the direction parallel to the stalk until the bacteria solution overflows from the top of the stalk, wherein 24 plants are injected, 3 tillers are injected for each plant, each variety is provided with three repeated samples, and the incidence condition is observed every day and recorded in time.

Calculation of incidence rate and disease index: performing follow-up investigation on the incidence condition after inoculation of injection, about 4 weeks later, investigating the incidence rate of panicles and the discoloration ratio of grains (the percentage of red-brown grains per panicle to the total number of grains), and calculating the disease index according to the standards of levels in Table 1.

Incidence rate of panicles=(number of diseased panicles/total number of inoculated panicles)*100%.

Disease index=[Σ(number of panicles of all levels*corresponding level number)/(total number of investigated panicles*highest level number)]*100.

After inoculation of rice of which the variety is Huazhan with stalk injection at the booting stage for bacterial panicle blight of rice provided in this embodiment, the diseased panicles show typical symptoms of panicle blight: red-brown disease spot on grains, inadequate grain filling/immature husk (FIG. 2A); the incidence rate is 100% (FIG. 2B), and the disease index is up to 76 (FIG. 2C).

Specifically, by adopting the method of stalk injection at the booting stage, the inoculation test of bacterial panicle blight of rice is performed on Yongyou potted under natural conditions and Huazhan planted in the field. The test results show that no matter in pot planting environment or field planting environment, the inoculation method of stalk injection at the booting stage shows high incidence rate, typical symptom and severe incidence condition, is a simple, convenient and efficient inoculation method, can be widely used for the identification and evaluation of resistance of panicle blight, the pathogenic typing of pathogenic bacteria, and the research on the interaction between pathogenic bacteria and host, and provides a powerful technical guarantee for prevention and control of the disease.

The present invention is exemplarily described above in combination with the drawings. Obviously, the implementation of the present invention is not limited by the above modes. Various improvements made by adopting the method ideas and technical solutions of the present invention or the ideas and technical solutions of the present invention directly applied to other occasions without improvements shall be within the protection scope of the present invention.

We claim:

1. An artificial inoculation method for bacterial panicle blight of rice, comprising the following steps:
S1. preparing an inoculum using a bacteria solution of Burkholderia glumae;
S2. inoculating at the booting stage of rice, wherein when booting, the stable and closed environment in the stalk can provide sufficient nutrients and high humidity for the colonization of pathogenic bacteria, which is conducive to the incidence of disease;
S3. adopting an inoculation method of injection, selecting the hollow booting part of a rice stalk, and injecting the inoculum prepared in the step S1 into the stalk from bottom to top by an injector; and
S4. after inoculation, according to the number of diseased panicles and the number of diseased grains, recording the incidence condition of rice, and calculating the incidence rate and disease index.

2. The artificial inoculation method for bacterial panicle blight of rice according to claim 1, wherein the inoculum in the step S1 is acquired by the following steps:
S11. transferring standard strains of Burkholderia glumae frozen at −70° C. into a NA liquid medium containing 50 μg/mL ampicillin in a ratio of 1:1000;
S12. culturing for 16-24 h at 37° C. and 200 rpm/min; and
S13. diluting the bacteria solution cultured in the step S12 with sterile purified water until the OD600 value of the bacteria solution is 0.2-0.3.

3. The artificial inoculation method for bacterial panicle blight of rice according to claim 1, wherein in the step S3, the specific position of injection is 5-7 cm below the last leaf of rice.

4. The artificial inoculation method for bacterial panicle blight of rice according to claim 1, wherein in the step S3, the injection volume of the inoculum is 0-1 mL until the bacteria solution overflows from the top of the stalk.

5. The artificial inoculation method for bacterial panicle blight of rice according to claim 1, wherein in the step S3, the inoculation is performed in an environment where the average daily temperature is greater than 30° C. and the relative humidity is greater than 75%.

6. The artificial inoculation method for bacterial panicle blight of rice according to claim 1, wherein in the step S3, the inoculation is specifically performed between 16:00-18:00 in the evening.

7. The artificial inoculation method for bacterial panicle blight of rice according to claim 4, wherein the injection volume of the inoculum is 0.5 mL.

8. The artificial inoculation method for bacterial panicle blight of rice according to claim 1, wherein in the step S3, at least 3 tillers are injected for each rice plant.
